(19) United States
(12) Patent Application Publication
(10) Pub. No.: US 2014/0235715 A1
(43) Pub. Date: Aug. 21, 2014

(54) TASTE MASKING FORMULATIONS OF FATTY ACIDS

(71) Applicant: Eric Kuhrts, Novato, CA (US)

(72) Inventor: Eric Kuhrts, Novato, CA (US)

(21) Appl. No.: 14/261,722

(22) Filed: Apr. 25, 2014

Related U.S. Application Data
(63) Continuation of application No. 13/663,087, filed on Oct. 29, 2012, now abandoned.

(51) Int. Cl. A61K 47/44; A61K 47/08; A23L 2/52

(52) U.S. Cl. CPC A61K 47/44; A23L 2/52; A61K 47/08
USPC 514/549; 514/552; 514/560

(57) ABSTRACT

Methods and formulations for improving the sensory characteristics and stability of dietary fatty acids for use in beverages, liquid concentrates, or other formulations are disclosed.

This application is a continuation application of U.S. patent application Ser. No. 13/663,087, filed Oct. 29, 2012, which claims the benefit of U.S. Provisional Application No. 61/553,177 filed on Oct. 29, 2011, each of which is incorporated herein by reference.

### FIELD OF THE INVENTION

The present invention relates to unique compositions comprising pleasant tasting and smelling, water-soluble formulations of dietary or nutritional fatty acids.

### BACKGROUND

Dietary or nutritional fatty acids are a family of unsaturated fatty acids that include the omega-3 fatty acids such as eicosapentaenoic acid (EPA) and docosahexaenoic acid (DHA), as well as omega-6 and omega-9 fatty acids. One of the primary sources for the omega-3 fatty acids is fish oil; however, omega-3 fatty acids can also be obtained from botanical sources and algae. The cardiovascular and other health benefits are now well known, in addition to their importance in nutrition. For example, consumption of nutritional or dietary fatty acids have been identified with many health benefits, having the potential to impact numerous diseases such as cardiovascular, neurological, immune function, and arthritis. Due to the increased awareness of the health benefits of the omega-3 class of fatty acids, dietary food supplements of fish oil or flax oil have become popular. With the availability of high quality fish oils, it is now possible to make beverages containing omega-3 fatty acids, or fish oil, but the low solubility and the fishy taste of the oils remains a serious problem for consumer acceptance.

### SUMMARY

Due to the many desirable properties of nutritional or dietary fatty acids, such as fish oil, it would be advantageous to have a pleasant tasting, water-soluble formulation of these fatty acids for use in beverages. Such a product would have more desirable sensory qualities for consumers.

Thus, in one aspect, the present disclosure provides a water-soluble formulation, comprising dietary fatty acid, non-ionic surfactant, lipophilic taste masking agent, and water.

In another example, a method of taste masking dietary fatty acid in water can comprise steps of warming non-ionic surfactant to a temperature, and combining dietary fatty acid with the non-ionic surfactant, lipophilic taste masking agent, and water to form stabilized, clear, water-soluble, fatty acid solution. In a specific example, the step of combining can be further characterized by combining the dietary fatty acid, the non-ionic surfactant after warming, and the lipophilic taste masking agent to form a surfactant-dietary fatty acid-lipophilic taste masking agent mixture; and combining the surfactant-dietary fatty acids-lipophilic taste masking mixture with the water.

In another example, a method of making a pleasant tasting and smelling, water-soluble pharmaceutical liquid composition of dietary fatty acid can comprise multiple steps. The steps can include heating water-soluble non-ionic surfactant in a container to a temperature of about 90° F. to about 200° F. while mixing the surfactant until clear; adding dietary fatty acid triglyceride to the non-ionic surfactant and stirring until thoroughly mixed so as to constitute from 0.1 wt % to 25 wt % dietary fatty acid and from 70 wt % to 99.9 wt % non-ionic surfactant, wherein the dietary fatty acid is sufficiently dispersed or dissolved in the non-ionic surfactant so that a gel composition is formed that contains no visible micelles or particles of dietary fatty acid; dissolving lipophilic essential oil or taste masking agent in said gel composition; and adding the gel composition containing the lipophilic essential oil or taste masking agent to warm water while continuously stirring the water until a clear solution is formed.

### DETAILED DESCRIPTION

Reference will now be made to the examples illustrated, and specific language will be used herein to describe the same. It will nevertheless be understood that no limitation of the scope of the technology is thereby intended. Additional features and advantages of the technology will be apparent from the detailed description which follows, which illustrate, by way of example, features of the technology.

### I. Definitions

The abbreviations used herein have their conventional meaning within the chemical and biological arts.

“Pharmaceutically acceptable salts” or “salts” include salts of the active compounds which are prepared with nontoxic acids or bases, depending on the particular substituent moieties found on the compounds described herein. When formulations of the present disclosure contain acidic functionalities, base addition salts can be obtained by contacting the neutral form of such compounds with a sufficient amount of the desired base, either neat or in a suitable inert solvent. Examples of pharmaceutically acceptable base addition salts include sodium salts, potassium salts, calcium salts, ammonium salts, organic amino salts, magnesium salts, and the like. When formulations of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids including, but not limited to, hydrochloric acid, hydrobromic acid, nitric acid, carbonic acid, monohydrogencarbonic acid, phosphoric acid, monohydrogenphosphoric acid, dihydrogenphosphoric acid, sulfuric acid, monohydrogensulfuric acid, hydriodic acid, phosphorous acids and the like, as well as the salts derived from relatively nontoxic organic acids including, but not limited to, acetic acid, propionic acid, isobutyric acid, maleic acid, malonic acid, benzoic acid, succinic acid, suberic acid, fumaric acid, lactic acid, mandelic acid, phthalic acid, benzenesulfonic acid, p-tolylsulfonic acid, citric acid, tartaric acid, methanesulfonic acid, and the like. Also included are salts of amino acids such as arginate and the like, and salts of organic acids such as glucuronic or galactunoric acids and the like (see, for example, Berge et al., “Pharmaceutical Salts”, Journal of Pharmaceutical Science, 1977, 66, 1-19). Certain specific formulations of the present disclosure contain both basic and acidic functionalities that allow the compounds to be converted into either base or acid addition salts. The neutral forms of the compounds can be regenerated by contacting the salt with a base or acid and isolating the parent compound in the conventional manner. The parent form of the compound differs from the various salt forms in certain physical properties, such as solubility in polar solvents.

“Dietary fatty acid(s)” as used herein includes nutritional fatty acids, omega-3 fatty acids derived from natural sources such as fish, algae or vegetable sources, including botanical sources such as chia, sage, Salvia hispanica, or flax sources derived from linseed, or produced synthetically. The following is a list of omega-3 fatty acids (Table 1) followed by a list of botanical extracts of omega-3 fatty acids (Table 2). These lists are exemplary only, and are not considered to be limiting.

TABLE 1
List of several common n-3 fatty acids found in nature

| Common Name | Lipid Name | Chemical Name |
|---|---|---|
| — | 16:3 (n-3) | all-cis-7,10,13- |
| | | hexadecatrienoic acid |
| Alpha-Linolenic acid (ALA) | 18:3 (n-3) | all-cis-9,12,15- |
| | | octadecatrienoic acid |
| Stearidonic acid (STD) | 18:4 (n-3) | all-cis-6,9,12,15- |
| | | octadecatetraenoic acid |
| Eisosatrienoic acid (ETE) | 20:3 (n-3) | all-cis-11,14,17- |
| | | eicosatrienoic acid |
| Eicosatetraenoic acid (ETA) | 20:4 (n-3) | all-cis-8,11,14,17- |
| | | eicosatrienoic acid |
| Eicosapentaenoic acid (EPA) | 20:5 (n-3) | all-cis-5,8,11,14,17- |
| | | eicosapentaenoic acid |
| Docosapentaenoic acid (DPA), | 22:5 (n-3) | all-cis-7,10,13,16,19- |
| Clupanodonic acid | | docosapentaenoic acid |
| Docosahexaenoic acid (DHA) | 22:6 (n-3) | all-cis-4,7,10,13,16,19- |
| | | docosahexaenoic acid |
| Tetracosapentaenoic acid | 24:5 (n-3) | all-cis-9,12,15,18,21- |
| | | docosahexaenoic acid |
| Tetracosahexaenoic acid | 24:6 (n-3) | all-cis-6,9,12,15,18,21- |
| (Nisinic Acid) | | tetracosenoic acid |

TABLE 2
Sources of botanical extracts of omega-3 fatty acids

| Common Name | Alternative Name | Linnaean Name | % n-3 |
|---|---|---|---|
| Chia | Chia sage | Salvia hispanica | 64 |
| Kiwifruit | Chinese gooseberry | Actinidia chinensis | 62 |
| Perilla | Shiso | Perilla frutescens | 58 |
| Flax | Linseed | Linum usitatissimum | 55 |
| Lingonberry | Cowberry | Vaccinium vitis-idaea | 49 |
| Camelina | Gold-of-pleasure | Camelina sativa | 36 |
| Purslane | Portulaca | Portulaca oleracea | 35 |
| Black Raspberry | — | Rubus occidentalis | 33 |

Dietary Fatty Acids containing omega-3 fatty acids may also be derived from algae such as Crypthecodinium cohnii and Schizochytrium, which are rich sources of DHA, or brown algae (kelp) for EPA. “Dietary fatty acid(s)” may also include conjugated linoleic acid (CLA), omega-6 fatty acids, and omega-9 fatty acids, such as linolenic acid, linoleic acid (18:2), and gamma linolenic acid (GLA, 18:3). Vegetarian polyunsaturated omega-3 fatty acid pre-cursors, such as stearidonic acid, are also included under the definition of “dietary fatty acids.” Stearidonic acid, for example, is a pre-cursor to eicosapentaeonoic acid (EPA) in humans. Dietary fatty acids such as fish oil omega-3 fatty acids can exist as free fatty acids, ethyl esters, and triglycerides. In this invention, the triglyceride form is the most preferred, as surprisingly, it results in the most clear aqueous solution with the least formation of solid gels, or milky opaque solutions.

“Essential oils” are concentrated hydrophobic (water-hating) liquids that consist primarily of volatile aroma compounds from plants. Essential oils are generally extracted from plant material by distillation, or solvent extraction. Some common essential oils include; clove, sweet orange, lemon, spearmint, lavender, peppermint, and eucalyptus, but also include many other diverse botanical oils such as nutmeg, cumin, and jasmine. These essential oils are lipophilic, or oil loving, so they are not miscible or soluble in water. However in this invention, these essential oils can be incorporated into a water soluble system with other oils such as dietary fatty acids, to help taste mask objectionable flavors and odors, and make them water-soluble. One of the unique features of essential oils, once they have been distilled or extracted and purified to high concentrations, is their effectiveness for this purpose at very low concentrations. Once made water soluble in the formulations in the instant invention, these oils provide effective taste and odor masking at levels in the parts per million (ppm). One “essential oil” that is very effective for this purpose is Clove oil (Syzygium aromaticum L.). Clove oil has been used as a spice to flavor a number of food preparations and recipes. The major essential oil in clove is eugenol, which consists of about 80% of the essential oil content. Clove is known to have antimicrobial, antiseptic, anticarcinogenic, and antioxidative properties. Clove has also been used as a home remedy for dental pain relief from toothache, and in aromatherapy. Another essential oil that can be effective for use herein is eugenol, which can be derived from clove oil, nutmeg, cinnamon, basil, or bay leaf.

A “non-ionic surfactant,” as used herein, is a surface-active agent that tends to be non-ionized (i.e. uncharged) in neutral solutions (e.g. neutral aqueous solutions).

The terms “patient’ and “subject” are used interchangeably, and refer to humans and other mammals. In one specific example, the patient or subject is human.

As used herein, the term “titration” means the slow addition of a compound or solution to a liquid while mixing. The rate at which the compound or solution is added should not exceed a certain threshold, or the clear nature and viscosity of the solute is lost. Slow addition can be as a drizzle or drop by drop, but in no case will typically equal large volumes. Slow addition can be specified as a percent of the volume it is being added to per second or per minute, for example 5 mL per second to 100 mL water, or 5 wt % addition per second or minute of the content being added to water or water containing beverage.

As used herein, the term “clear aqueous solution” in reference to a solution or even a very fine dispersion containing dietary fatty acid means a water containing solution (e.g. a beverage) that is free of visible particles of undissolved dietary fatty acid. In accordance with some embodiments, the clear aqueous solution is not a more traditional dispersion or suspension, and remains clear upon sitting undisturbed for 1 hour or more. Often, very small micelles are formed that are not visible, and thus, the “solution” is clear.

The term “water-soluble” herein refers to the solubilization or very fine dispersion of dietary fatty acids so that they are not visible to the naked eye in solution. Often, in the formulations of the present disclosure, the fatty acids can form micelles in water with a non-ionic surfactant barrier, and the micelles can be smaller than about 100 nm in size, and often are about 15 nm to about 30 nm in size. Thus, whether the dietary fatty acids are strictly dissolved or merely so finely dispersed that the solution they form within is clear, this is still considered to be “water-soluble” in accordance with embodiments of the present disclosure.

As used herein, the term “oxidation” refers particularly to the degradation or spoiling of an oil or fat through exposure to air or oxygen, resulting in a loss of electrons or an increase in oxidation state. Oxidation can be the result of different chemical mechanisms during the processing, storage, or heating of an oil or fat. There are various types of oxidation, namely autooxidation, photosensitized oxidation, thermal oxidation, and enzymatic oxidation. One type of oxidation particularly relevant in the context of the present disclosure is thermal oxidation, because the formulations and process involved in this application involve heating, and thermal oxidation is one of the most rapid forms of oxidation. Various types of oxidation products are produced by autooxidation and thermal oxidation, such as hydroperoxides, aldehydes, and ketones. These degradation products can be measured, providing an analytical index for aging or stability studies for various oils under different conditions, providing a comprehensive spectrum of decomposition products.

As used herein, the term “peroxide value” or “PV” refers to a quantitative measure of the oxidation of oil. Peroxide value is usually given in meq/Kg of oil (milliequivalents per kilogram). One method used to determine PV is American Oil Chemists' Society Official Method (AOCS) Cd 8-53. The peroxide value is also a means of assessing the extent of rancidity reactions that have occurred during storage of a fat or oil. Peroxide value is defined as the amount of peroxide oxygen per kilogram of oil. Peroxide value is measured by determining the amount of iodine which is formed by the reaction of peroxides formed in the oil with iodide ion. A decrease in peroxide values leads to better sensory characteristics or quality of the oil, such as smell and taste. An acceptable peroxide value is that which is beneath about 40. In a stricter example, acceptable peroxide values can be less than about 30, less than about 20, and preferably less than about 15.

Concentrations, amounts, solubilities, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a concentration range of 0.5 to 400 should be interpreted to include not only the explicitly recited concentration limits of 0.5 and 400, but also to include individual concentrations within that range, such as 0.5, 0.7, 1.0, 5.2, 8.4, 11.6, 14.2, 100, 200, 300, and sub-ranges such as 0.5-2.5, 4.8-7.2, 6-14.9, 55, 85, 100-200, 117, 175, 200-300, 225, 250, and 300-400, etc. This interpretation should apply regardless of the breadth of the range or the characteristic being described.

### II. Water Soluble Formulations

Benefits may be realized from adding nutritional fatty acids such as omega-3 fish oils, algae derived DHA, conjugated linoleic acid (CLA, C18:2), flax, chia oil, etc., to beverages. Until recently, highly purified and concentrated fish oils have not been available. With the availability of these purified fish oils that are molecularly distilled, it is possible to make beverages containing omega-3 fatty acids from fish oil or algae, but the taste and solubility of the oils can be a serious issue. Normally, these oils are kept frozen to prevent or slow down oxidation. As soon as these oils are defrosted and processed, they begin to undergo oxidation. Oxidation is a natural process that occurs when oils are exposed to air or oxygen. The oxidation of oils can be measured quantitatively by measuring certain markers of oxidation such as the peroxide value (PV) or isoprostanes. Rancidification is the oxidation of fats, fatty acids, or edible oils, and most people are familiar with the term rancid to describe the change in smell associated with edible oils or fats such as butter after exposure to air for prolonged periods. A rancid oil or fat also has an objectionable taste. Oxidation is the loss of electrons or increase in oxidation state by a molecule, atom, or ion.

Once oxidized, the undesirable sensory characteristics become apparent. Odor and taste are directly correlated with oxidation. For example, the fishy odor and taste of fish oil is a highly undesirable property of a fish oil-containing beverage. It would be desirable to have a formulation of nutritional fatty acids that were soluble in water containing beverages. It would also be desirable to provide a water-soluble omega-3 fish oil (or other dietary fatty acid) formulation that would taste acceptable, or be virtually free of undesirable odor and taste. In addition, it would also be advantageous to have a process or method of making such formulations.

It has been discovered that non-ionic surfactants may be used to increase the solubility and/or bioavailability of dietary fatty acids, as well as to solubilize a taste masking component to be incorporated therein that is fat (lipid) soluble. Thus, non-ionic surfactants may be used to form water-soluble formulations containing dietary fatty acids and a fat soluble taste masking component. The taste masking component to be used is provided in the formulation at a very subtle level, and can be incorporated into the micelle when an aqueous solution is mixed properly with the surfactant, the fatty acid, and the taste masking agent. Essential oils are acceptable candidates for this purpose, as they are normally not soluble in water, and are hydrophobic and lipophilic. Essential oils also are very potent at very low concentrations (potent in terms of taste and odor). One lipophilic taste masking agent or compound that is suited for this application is the essential oil from clove, which contains eugenol. Clove oil is produced from S. aromaticum, and is available in the following: bud oil, derived from the flower-buds containing about 60-90% eugenol; leaf oil, derived from the leaves containing 82-88% eugenol; and stem oil, derived from the twigs containing 90-95% eugenol, to name a few.

In one aspect, the present disclosure provides a water-soluble formulation including dietary fatty acid, non-ionic surfactant, and taste masking agent. In one example, the taste masking agent can be clove oil, or can contain eugenol (4-allyl-2-methoxyphenol), the principle essential oil derived from clove. In other embodiments, the water-soluble formulation can be formulated in the absence of an alcohol, e.g. the dietary fatty acid formulation is not first dissolved in alcohol and then added to water. Thus, in some embodiments, the water-soluble formulation is a non-alcoholic formulation. A “non-alcoholic” formulation, as used herein, is a formulation that does not include (or includes only in trace amounts) methanol, ethanol, propanol, butanol, or other alcoholic solvents. In other embodiments, the formulation does not include (or includes only in trace amounts) ethanol.

As mentioned, non-ionic surfactants used herein include surface-active agents that tends to be non-ionized (i.e. uncharged) in neutral solutions (e.g. neutral aqueous solutions). Useful non-ionic surfactants include, for example, non-ionic water soluble mono-, di-, and tri-glycerides; non-ionic water soluble mono- and di-fatty acid esters of polyethyelene glycol; non-ionic water soluble sorbitan fatty acid esters (e.g. sorbitan monooleates such as SPAN 80 and TWEEN 20 (polyoxyethylene 20 sorbitan monooleate)); polyglycolyzed glycerides; non-ionic water soluble triblock copolymers (e.g. poly(ethyleneoxide)/poly-(propyleneoxide)/poly(ethyleneoxide) triblock copolymers such as POLOXAMER 406 (PLURONIC F-127), and derivatives thereof.

Examples of non-ionic water soluble mono-, di-, and tri-glycerides include propylene glycol dicarpylate/dicaprate (e.g. MIGLYOL 840), medium chain mono- and diglycerides (e.g. CAPMUL and IMWITOR 72), medium-chain triglycerides (e.g. caprylic and capric triglycerides such as LAVRAFAC, MIGLYOL 810 or 812, CRODAMOL GTCC-PN, and SOFTISON 378), long chain monoglycerides (e.g. glyceryl monooleates such as PECEOL, and glyceryl monolinoleates such as MAISINE), polyoxyl castor oil (e.g. macrogolglycerol ricinoleate, macrogolglycerol hydroxystearate, macrogol cetostearyl ether), polyethylene glycol 660 hydroxystearate and derivatives thereof.

Non-ionic water soluble mono- and di-fatty acid esters of polyethyelene glycol include d-α-tocopheryl polyethyleneglycol 1000 succinate (TPGS), poyethyleneglycol 660 12-hydroxystearate (SOLUTOL HS 15), polyoxyl oleate and stearate (e.g. PEG 400 monostearate and PEG 1750 monostearate), and derivatives thereof.

Polyglycolyzed glycerides include polyoxyethylated oleic glycerides, polyoxyethylated linoleic glycerides, polyoxyethylated caprylic/capric glycerides, and derivatives thereof. Specific examples include LABRAFIL M-1944CS, LABRAFIL M-2125CS, LABRASOL, SOFTIGEN, and GELUCIRE.

In some embodiments, the non-ionic surfactant is a macrogolglycerol hydroxystearate (polyoxyl castor oil, glycerol-polyethylene glycol oxystearate), or derivative thereof. These compounds may be synthesized by reacting either castor oil or hydrogenated castor oil with varying amounts of ethylene oxide. Macrogolglycerol ricinoleate is a mixture of 83% relatively hydrophobic and 17% relatively hydrophilic components. The major component of the relatively hydrophobic portion is glycerol polyethylene glycol ricinoleate, and the major components of the relatively hydrophilic portion are polyethylene glycols and glycerol ethoxylates. Macrogolglycerol hydroxystearate (glycerol-polyethylene glycol oxysterate) is a mixture of approximately 75% relatively hydrophobic of which a major portion is glycerol polyethylene glycol 12-oxystearate.

In some embodiments, the water-soluble formulations include the dietary fatty acid, and macrogolglycerol hydroxystearate, to form a transparent water-soluble formulation. A “transparent water-soluble formulation,” as disclosed herein, refers to a formulation that can be clearly seen through with the naked eye and is optionally colored. In some embodiments, the transparent water-soluble formulations do not contain particles (e.g. particles of undissolved dietary fatty acid) visible to the naked eye. Thus, in some embodiments, the transparent water-soluble formulations are not opaque, cloudy or milky-white. Transparent water-soluble formulations disclosed herein do not include milky-white emulsions or suspensions in vegetable oil such as corn oil. Transparent water-soluble formulations are also typically not formed by first dissolving the dietary fatty acid in alcohol, or other organic solvents, and then mixed with water.

In some embodiments, the formulation is a non-aprotic solvated formulation. The term “non-aprotic solvated,” as used herein, means that water soluble aprotic solvents are absent or are included only in trace amounts. Water soluble aprotic solvents are water soluble non-surfactant solvents in which the hydrogen atoms are not bonded to an oxygen or nitrogen and therefore cannot donate a hydrogen bond.

In some embodiments, the water-soluble formulation does not include (or includes only in trace amounts) a polar aprotic solvent. Polar aprotic solvents are aprotic solvents whose molecules exhibit a molecular dipole moment but whose hydrogen atoms are not bonded to an oxygen or nitrogen atom. Examples of polar aprotic solvents include aldehydes, ketones, dimethyl sulfoxide (DMSO), and dimethyl formamide (DMF). In other embodiments, the water soluble formulation does not include (or includes only in trace amounts) dimethyl sulfoxide. Thus, in some embodiments, the water soluble formulation does not include DMSO. In a related embodiment, the water soluble formulation does not include DMSO or ethanol.

In still other embodiments, the water-soluble formulation does not include (or includes only in trace amounts) a non-polar aprotic solvent. Non-polar aprotic solvents are aprotic solvents whose molecules exhibit a molecular dipole of approximately zero. Examples include hydrocarbons, such as alkanes, alkenes, and alkynes.

In some embodiments, the water-soluble formulation consists essentially of dietary fatty acid, non-ionic surfactant, and a taste masking agent. That is, the formulation does not include any water, but optionally may include additional components widely known in the art to be useful in neutraceutical formulations, such as preservatives, taste enhancers, colors, buffers, water, etc. In these formulations, a fat-soluble taste masking compound can be dissolved in the surfactant/fatty acid triglyceride or oil mixture. Examples of fatty acid triglycerides are CLA or 80% conjugated linoleic acid with a triglyceride content of 90% as measured by GC (gas chromatography), or an omega-3 fish oil which is 65% total omega-3 with a total triglyceride content of 96%. Any fatty acid with a high content of triglycerides, as opposed to ethyl esters (EE) is preferred.

In some embodiments, the water-soluble formulation is a water-solubilized formulation, i.e. it includes a dietary fatty acid, a fat soluble taste masking compound, a non-ionic surfactant, and water (e.g. a water-containing liquid) but does not include organic solvents (e.g. ethanol). For example, the formulation can be in a non-alcoholic form, and in other examples, does not include any other solvents other solvents at all (other than water when admixed therewith to form a liquid beverage, concentrate, etc.). The surfactant/fatty acid/fat soluble taste masking agent/water complex can self-assemble into micelles, once a critical concentration is reached. These micelles are invisible to the naked eye, so that, in some embodiments, the water-solubilized formulation is a transparent water-soluble formulation.

Though certain formulation relative percentages will be set forth below in further detail, in one specific example, one formulation can comprise about 40 to 80 vol % non-ionic surfactant, about 2 to 50 vol % dietary fatty acid, about 0.001 to 1 vol % lipophilic taste masking agent, and can be devoid of water (i.e. prior to adding to water). In another example, the formulation can comprises about 10 to 40 vol % non-ionic surfactant, about 0.5 to 20 vol % dietary fatty acid, about 0.0005 to 0.5 vol % lipophilic taste masking agent, and about 50 to 85 vol % water.

### III. Methods

In another aspect of the present disclosure is described a method of producing more stable, water-soluble fatty acid formulations with better taste and smell characteristics and shelf life. This is especially helpful for fish oils, where oxidation will result in a fishy smell and taste, as described or characterized by high PV values defined previously. Thus, if proper procedure is not followed, a semi-solid gel-like, cloudy or milky, high viscosity solution may be obtained that is not desirable. This waxy, cloudy, high viscosity gel is not suitable for forming clear solutions in water or beverages. Rather, it becomes a solidified milky white mass. In contrast, by slowly titrating or adding the dietary fatty acid, warm non-ionic surfactant, and the lipophilic taste masking agent formulation to warm water, a clear solution can be obtained. In one example, the non-ionic surfactant and/or water are within a certain pre-described temperature ranges, e.g., from 80 to 200° F. for either. Typically, if the resulting dietary fatty acid/surfactant gel mixture is then added to the water too fast, a solid gel-like mass can result. In a particular embodiment, the dietary fatty acid gel is added to water at a rate of from about 0.05 mL/sec to about 25.0 mL/sec. In another particular embodiment, the temperature of the non-ionic surfactant does not exceed 200° F., e.g., from 80 to 200° F., and is typically maintained at a temperature of 90 to 120° F. The non-ionic surfactant can be stirred thoroughly to remove bubbles (oxygen), and until clear.

In a particular embodiment, once the dietary fatty acid has been added to the surfactant, it is stirred for at least 10 minutes, or more, and then a small amount of lipophilic taste masking agent, such as clove oil or other essential oil, is added to the surfactant/fatty acid mixture, and then stirring can be continued for about 1 hour or until thoroughly mixed and stabilized. The amount of essential oil that is added can be very small, e.g., a fraction of a mL per liter of final volume. More specifically about 2 to 200 microliters (μL) per liter of dietary fatty acid/non-ionic surfactant can be used, or in another embodiment, from 30 to 100 microliters per liter, e.g. about 2-3 drops of clove oil per liter. In a more particular embodiment, the water to which the dietary fatty acid/non-ionic surfactant/lipophilic taste masking age (essential oil) it is to be added can be warmed as previously indicated. However, in one example, the warming can be from about 100 to 150° F. The temperature can likewise be maintained at a predetermined level, e.g., about 100° F., while slowly adding the dietary fatty acid mixture and mixed until a clear solution is formed.

In another aspect, the present disclosure provides for a more stable formulation of a liquid concentrate or beverage comprising dietary fatty acids, with a low peroxide value (PV), better shelf life characteristics, and enhanced consumer acceptance. For example, a beverage can be made from fish oil omega-3 fatty acids without a fishy odor or taste, or objectionable sensory qualities. In addition, stable formulations of dietary fatty acids and oils in liquid concentrates or beverages that do not need to be kept frozen to prevent oxidation or development of off taste or objectionable smell can also be prepared.

In another aspect, the present disclosure provides a method for enhancing the sensory characteristics of a dietary fatty acid. The method includes combining dietary fatty acids, and a non-ionic surfactant to form a surfactant-dietary fatty acid mixture, then combining this mixture with the essential oil, e.g., clove oil extract or eugenol-containing oil. Other botanical sources of eugenol such as holy basil (Ocimum sanctum), or Eugenia caryophyllata can also be used. Pure eugenol can also be used, such as 99% eugenol available from Sigma-Aldrich, CAS Number 97-53-0, synonym; 2-Methoxy-4-(2-propenyl)phenol, 4-Allyl-2-methoxyphenol, 4-Allylgualacol. The non-ionic surfactant/dietary fatty acid/essential oil/water mixture has better taste and smell characteristics during consumption and after storage (aging).

In another aspect, the present disclosure provides a method of dissolving a dietary fatty acid and fat soluble taste masking agent (essential oil), in water. The method includes combining dietary fatty acid with a non-ionic surfactant to form a surfactant-dietary fatty acid mixture. The essential oil or fat soluble (lipophilic) taste masking agent is then added to the surfactant-dietary fatty acid mixture and then mixed with water, thereby dissolving the dietary fatty acid and taste masking agent in water. The surfactant dietary fatty acid and taste masking mixture is typically not added at a rate to exceed 5 mL per second to a volume of water of 100 mL, or not more than 5% of the volume of water per second of the volume of water it is being added to. When adding the non-ionic surfactant/dietary fatty acid/lipophilic taste masking agent to water, the water is to be stirred or otherwise admixed continuously. Additionally, the water can be heated to increase solubility during this process. The heating temperature is typically selected to avoid chemical breakdown of the dietary fatty acid and/or non-ionic surfactant. The temperature of the dietary fatty acid emulsion (dietary fatty acid/non-ionic surfactant/lipophilic taste agent) typically will not exceed 150° F., and the water temperature also will not typically exceed 150° F., though temperatures outside this range are also usable as described previously. More typically, however, the temperature of both can be maintained at between 100 and 120° F. In some embodiments, the resulting solution is a water-soluble formulation or transparent water soluble formulation as described above. For example, the resulting solution may be a water soluble formulation that is a crystal clear solution, with no particles visible to the naked eye.

### IV. Dosages and Dosage Forms

The amount of dietary fatty acid adequate to treat a disease is defined as an “effective amount” or a “therapeutically effective dose.” In accordance with this, methods of treating a subject for a disease can be carried out using an effective amount or pharmaceutically effective dose a water soluble formulation such as those described herein. In some embodiments, the subject is a mammalian subject, such as a human or domestic animal.

The dosage schedule and amounts effective for this use, i.e., the “dosing regimen,” will depend upon a variety of factors, including the stage of the disease or condition, the severity of the disease or condition, the general state of the patient's health, the patient's physical status, age and the like. In calculating the dosage regimen for a patient, the mode of administration also is taken into consideration.

The dosage regimen also takes into consideration pharmacokinetics parameters well known in the art, i.e., the rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; the latest Remington's, supra). The state of the art allows the clinician to determine the dosage regimen for each individual patient and disease or condition treated.

Single or multiple administrations of dietary fatty acid formulations can be administered depending on the dosage and frequency as required and tolerated by the patient. The formulations should provide a sufficient quantity of active agent to effectively treat the disease state. Lower dosages can be used, particularly when the drug is administered to an anatomically secluded site in contrast to administration orally, into the blood stream, into a body cavity or into a lumen of an organ. Substantially higher dosages can be used in topical administration. Actual methods for preparing parenterally administrable dietary fatty acid formulations will be known or apparent to those skilled in the art and are described in more detail in such publications as Remington's, supra.

According to an embodiment, the dietary fatty acid triglyceride is present in the water-soluble formulation at a minimum concentration of from about 0.01% to about 35% by weight in the non-ionic surfactant to make the emulsion phase (including the presence of the lipophilic taste masking agent), or 1 to 10 wt % in the aqueous phase (when added to water). The lipophilic taste masking agent or essential oil, such as clove oil, can be present at any desirable level, but in one example, at a level of 2 to 200 μL/L (2-200 ppm) in the aqueous phase, an in another example, from 2 to 100 μL/L (2-100 ppm) in the aqueous phase. In another embodiment the dietary fatty acid can be present in the water-soluble formulation at a concentration from 1 wt % to 10 wt %, and the taste masking lipophilic agent, such as clove oil, can be present at a level of 5 to 50 μL/L (5-50 ppm), and the water is present at a level of 50-70 wt %. In a still more specific embodiment, the dietary fatty acid can be present in the water-soluble formulation at a concentration from 15 wt % to 30 wt % in the emulsion, or more specifically from 20 wt % to 30 wt %, or still more specifically from 25 wt % to 30 wt %, and the lipophilic taste masking agent, or clove oil, can be present in the emulsion at 5 to 50 μL/L (5-50 ppm) in the emulsion.

The dietary fatty acid triglycerides may also be present (e.g. in a beverage formulation) at a concentration from 0.5 to 1,000 mg per 8 fluid oz. beverage, or around 1-100 mg per mL in a liquid concentrate, and the taste masking lipophilic agent or clove oil at a level of 2-200 ppm (or 2-200 ppm) in the finished beverage. In other embodiments, the dietary fatty acid can be present at a concentration from 0.01 mg/mL to 100 mg/mL. In an aspect of the embodiments herein, there can be a maximum concentration for achieving a crystal clear solution. Concentrations of dietary fatty acid triglycerides above 40% in an emulsion using glycerol-polyethylene glycol oxystearate (i.e. macrogoglycerol hydroxystearate) for example, as the surfactant, will no longer result in a crystal-clear solution in water. Therefore, for dietary fatty acids, the concentration range can be from 0.1% to 25% in the surfactant, or 0.01 mg/mL to 250 mg/mL, with the preferred concentration around 90 mg/mL. This represents a ratio of dietary fatty acid to surfactant of 1:4. In some concentrated formulations (e.g. a soft gel capsule formulation), dietary fatty acid may be present at about 1 to 100 mg/mL, or around 20 mg/mL, or at least 1 mg/mL.

In other embodiments, the lipophilic taste masking agent, e.g., clove oil or eugenol-containing essential oil, is present in the water-soluble beverage formulation in a minimum amount of from about 2 μL/L to about 200 μL/L, or 2 to 200 ppm. In another embodiment, the taste masking agent is present in the water-soluble formulation in an amount from about 50 to 100 μL/L, or 50-100 ppm. In a more specific embodiment, the taste masking agent can be present at from 60-80 μL/L, or 60-80 ppm, in the finished formulation. The clove oil can be present in an amount of from 2 to 100 μL in a solution of 500 mL of liquid, for example, 60 μL is dissolved in 150 mL of a warm non-ionic surfactant with 30 mL of fatty acid triglycerides, and this is added to 310 mL of warm water. The total volume of the water-soluble concentrate is then about 500 mL, so the level of taste masking agent would be about 60 ppm. It is recognized that when preparing a concentrate, values outside of this range will be present as a result of there being less water or no water present.

Formulation ranges can be found in the following table that are considered exemplary, as amounts outside of these ranges can also be used as previously set forth.

Compound Formulation Ranges: Per 1 Liter Batch

| | | Dietary Fatty | | |
|---|---|---|---|---|
| | Non-ionic | acid with tri- | Taste masking | |
| Compound | surfactant | glycerides | lipophilic agent | Water |
| Volume | 200-300 mL | 10-100 mL | 2-200 μL | 600-789 mL |

In some embodiments, the water-soluble formulation is in the form of a pharmaceutical composition. The pharmaceutical composition may include dietary fatty acid triglyceride, a non-ionic surfactant, a taste masking agent such as eugenol or clove oil, and a pharmaceutically acceptable excipient. After a pharmaceutical composition including dietary fatty acid triglyceride of the invention has been formulated in an acceptable carrier, it can be placed in an appropriate container and labeled for treatment of an indicated condition. For administration of dietary fatty acid, such labeling would include, e.g., instructions concerning the amount, frequency and method of administration.

Any appropriate dosage form is useful for administration of the water-soluble formulation of the present invention, such as oral, parenteral and topical dosage forms. Oral preparations include tablets, pills, powder, dragees, capsules (e.g. soft-gel capsules), liquids, lozenges, gels, syrups, slurries, beverages, suspensions, etc., suitable for ingestion by the patient. Examples of liquid formulations are drops, sprays, aerosols, emulsions, lotions, suspensions, drinking solutions, gargles, and inhalants. Also, the formulations described herein can be administered by inhalation, for example, intranasally. Additionally, the formulations of the present invention can be administered transdermally. The formulations can also be administered by intraocular, intravaginal, and intrarectal routes including suppositories, insufflation, powders and aerosol formulations (for examples of steroid inhalants, see Rohatagi, J. Clin. Pharmacol. 35:1187-1193, 1995; Tjwa, Ann. Allergy Asthma Immunol. 75:107-111, 1995). Thus, the formulations described herein may be adapted for oral administration.

For preparing pharmaceutical compositions from the formulations of the present invention, pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, binders, preservatives, tablet disintegrating agents, or an encapsulating material. Details on techniques for formulation and administration are well described in the scientific and patent literature, see, e.g., the latest edition of Remington's Pharmaceutical Sciences, Maack Publishing Co, Easton Pa. (“Remington's”).

Suitable carriers include magnesium carbonate, magnesium stearate, talc, sugar, lactose, pectin, dextrin, starch (from corn, wheat, rice, potato, or other plants), gelatin, tragacanth, a low melting wax, cocoa butter, sucrose, mannitol, sorbitol, cellulose (such as methyl cellulose, hydroxypropylmethyl-cellulose, or sodium carboxymethylcellulose), and gums (including arabic and tragacanth), as well as proteins such as gelatin and collagen. If desired, disintegrating or co-solubilizing agents may be added, such as the cross-linked polyvinyl pyrrolidone, agar, alginic acid, or a salt thereof, such as sodium alginate. In powders, the carrier is a finely divided solid, which is in a mixture with the finely divided active component. In tablets, the active component is mixed with the carrier having the necessary binding properties in suitable proportions and compacted in the shape and size desired.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations of the invention can also be used orally using, for example, push-fit capsules made of gelatin, as well as soft, sealed capsules made of gelatin and a coating such as glycerol or sorbitol. Push-fit capsules can contain dietary fatty acid mixed with a filler or binders such as lactose or starches, lubricants such as talc or magnesium stearate, and, optionally, stabilizers. In soft capsules, dietary fatty acid may be dissolved or suspended in suitable liquids, such as fatty oils, liquid paraffin, or liquid polyethylene glycol with or without stabilizers.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, beverages, and emulsions, for example, water or water/propylene glycol solutions. For parenteral injection, liquid preparations can be formulated in solution in aqueous polyethylene glycol solution.

Aqueous solutions and beverages suitable for oral use can be prepared by dissolving the active component in water and adding suitable colorants, flavors, stabilizers, and thickening agents as desired. Aqueous suspensions suitable for oral use can be made by dispersing the active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The formulations of the invention can be delivered transdermally, by a topical route, formulated as applicator sticks, solutions, suspensions, emulsions, gels, creams, ointments, pastes, jellies, paints, powders, and aerosols.

The formulations may be administered as a unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted according to the particular application and the potency of the active component. The composition can, if desired, also contain other compatible therapeutic agents.

Subject non-ionic surfactants may be assayed for their ability to solubilize dietary fatty acid using any appropriate method. Typically, a non-ionic surfactant is warmed and contacted with the lipophilic taste masking agent and dietary fatty acid triglyceride and mixed mechanically and/or automatically using a shaker, vortex, or sonicator device. Water may be optionally added, for example, where the dietary fatty acid triglyceride/taste masking agent and/or surfactant is in powder form, or a liquid concentrate is desired. The solution is heated to increase solubility. The heating temperature is selected to avoid chemical breakdown of the dietary fatty acid triglyceride, taste masking compound or non-ionic surfactant. In a particular example, the surfactant, taste masking agent, and dietary fatty acid triglyceride is not heated above 200° F., and typically not more than 150° F., or even 120° F.

The resulting solution may be visually inspected for colloidal particles to determine the ° of solubility of the dietary fatty acid. Alternatively, the solution may be filtered and analyzed to determine the ° of solubility. For example, a spectrophotometer may be used to determine the concentration of dietary fatty acid present in the filtered solution. Typically, the test solution is compared to a positive control containing a series of known quantities of pre-filtered dietary fatty acid solutions to obtain a standard concentration versus UV/vis absorbance curve. Alternatively, high performance liquid chromatography may be used to determine the amount of dietary fatty acid in solution. Micelles in a size range of from 10 to 100 nm can be measured by light scattering experiments. Typical sizes are from 10 to 50 nm for fatty acid self-assembled micelles formed by this invention.

High throughput solubility assay methods are well known in the art. Typically, these methods involve automated dispensing and mixing of solutions with varying amounts of non-ionic surfactants, dietary fatty acid, and optionally other co-solvents. The resulting solutions may then be analyzed to determine the ° of solubility using any appropriate method as discussed above.

For example, the Millipore MultiScreen Solubility filter plate® with modified track-etched polycarbonate, 0.4 μm membrane is a single-use, 96-well product assembly that includes a filter plate and a cover. The device is intended for processing aqueous solubility samples in the 100-300 μL volume range. The vacuum filtration design is compatible with standard, microtiter plate vacuum manifolds. The plate is also designed to fit with a standard, 96-well microtiter receiver plate for use in filtrate collection. The MultiScreen Solubility filter plate® has been developed and QC tested for consistent filtration flow-time (using standard vacuum), low aqueous extractable compounds, high sample filtrate recovery, and its ability to incubate samples as required to perform solubility assays. The low-binding membrane has been specifically developed for high recovery of dissolved organic compounds in aqueous media.

The aqueous solubility assay allows for the determination of dietary fatty acid solubility by mixing, incubating and filtering a solution in the MultiScreen Solubility filter plate. After the filtrate is transferred into a 96-well collection plate using vacuum filtration, it can be analyzed by Ultraviolet-visible (UV/Vis) spectroscopy to determine solubility. Additionally, LC/MS or HPLC can be used to determine compound solubility, especially for compounds with low UV/Vis absorbance and/or compounds with lower purity. For quantification of aqueous solubility, a standard calibration curve may be determined and analyzed for each compound prior to determining aqueous solubility.

Test solutions may be prepared by adding an aliquot of concentrated drug or compound. in one example, the solutions are mixed in a covered 96-well MultiScreen Solubility filter plate for 1.5 hours at room temperature. The solutions are then vacuum filtered into a 96-well, polypropylene, V-bottomed collection plate to remove any insoluble precipitates. Upon complete filtration, 160 μL/well are transferred from the collection plate to a 96-well UV analysis plate and diluted with 40 μL/well of acetonitrile. The UV/Vis analysis plate is scanned from 260-500 nm with a UV/Vis microplate spectrometer to determine the absorbance profile of the test compound.

Thus, one skilled in the art may assay a wide variety of non-ionic surfactants to determine their ability of solubilize dietary fatty acid compounds.

The terms and expressions which have been employed herein are used as terms of description and not of limitation, and there is no intention in the use of such terms and expressions of excluding equivalents of the features shown and described, or portions thereof, it being recognized that various modifications are possible within the scope of the invention claimed. Moreover, any one or more features of any embodiment of the invention may be combined with any one or more other features of any other embodiment of the invention, without departing from the scope of the invention. For example, the features of the formulations are equally applicable to the methods of treating disease states described herein. All publications, patents, and patent applications cited herein are hereby incorporated by reference in their entirety for all purposes. All percentages are in weight percentage unless stated or the context dictates otherwise to one skilled in the art.

### EXAMPLES

The examples below are meant to illustrate certain embodiments of the invention, and are not intended to limit the scope of the invention.

### Example 1

Water-soluble taste masking compositions of omega-3 fatty acid triglycerides were formulated containing the non-ionic surfactant macrogolglycerol hydroxystearate (Glycerol-Polyethylene glycol oxystearate) and clove oil. First, the non-ionic surfactant was heated to about 100° F. and stirred until clear and virtually no bubbles are apparent. 80 mL of omega-3 fatty acid fish oil triglyceride (90% triglycerides), containing 30% total omega-3 fatty acids at room temperature is very slowly added into the warm macrogolglycerol hydroxystearate until a clear slightly viscous emulsion was formed containing dissolved omega-3 fatty acid triglycerides (hereinafter referred to as “omega-3 gel formulation”). 100 μL of clove oil (3 drops) were added to the non-ionic surfactant/fatty acid emulsion. The omega-3 gel formulation consisted of macrogolglycerol hydroxystearate (300 mL) and 80 mL (80 grams) of omega-3 fatty acids, representing a concentration of 27% for the omega-3 fatty acids in the non-ionic surfactant, and 90 μL for the clove oil. In another vessel, 620 mL of water is warmed to a temperature of 100° F. The emulsion containing the surfactant, omega-3 fish oil fatty acid triglyceride, and clove oil is slowly added to the warm water until a clear solution is formed with no visible particles. The omega-3 fatty acid/surfactant mixture was slowly added, at a rate of about 1 mL per second to the 620 mL of warm water that was maintained as a mixing vortex with a stirrer at 50 RPM, and maintained at a temperature of about 100° F. until a crystal clear solution was formed. The water was continuously stirred during the addition phase and after, until a clear liquid was formed. This solution contained self-assembled micelles, invisible to the naked eye, containing omega-3 fatty-acids, surfactant, clove oil, and water. This solution was tested for taste and smell characteristics and found to have acceptable sensory qualities. A sample of the same omega-3 fatty acids used in this example without the clove oil had a fishy odor, and fishy taste.

Compound Formulation Ranges: Per 1 Liter Batch

| | macrogolglycerol | Omega-3 tri- | | |
|---|---|---|---|---|
| Compound | hydroxystearate | glyceride fish oil | Clove Oil | Water |
| Approx | 300 mL | 80 mL | 100 μL | 620 mL |
| Volume | | | | |

The aqueous omega-3 fatty acid formulation was analyzed by HPLC to verify content of total fatty acids. The same water soluble concentrate described above was added to apple juice, and taste tests were conducted. The omega-3 fatty acid/apple juice mixture contained 32 mg of omega-3 fatty acids per 8 oz. serving size, and none of the 8 subjects in the taste panel were able to identify the presence of fish oil, either by odor or taste.

### Example 2

The following formulation was prepared. 35 mL of DHA (docosahexaenoic acid) oil from algae was dissolved in 150 mL of warm macrogolglycerol hydroxystearate by mixing until a clear gel was formed. 2 μL of food grade eugenol oil (71.8% eugenol, 6.2% iso-eugenol) was added to this emulsion. In another vessel, 310 mL of warm water is prepared, to which is added the forgoing emulsion. The DHA/surfactant/eugenol emulsion was then very slowly added to the warm water which was mixing with a paddle suspended and rotating at 50 RPM by slowly adding as a drizzle, or drop-by-drop using a titration apparatus. The DHA oil is added very slowly to the mixing water to avoid solidification of the liquid into a solid gel, or cloudy white mass. The DHA oil was added at the rate of 1 mL every 10 seconds or more while stirring continues. A clear solution was formed with no visible particles or micelles. This taste masked, water soluble DHA fatty acid solution was tested and found to have acceptable taste and smell characteristics when compared to the DHA oil in the same formula without the eugenol oil.

### Example 3

50 mL of a conjugated linoleic acid (CLA) consisting of 90% triglyceride content is added to 50 mL of warm macrogolglycerol hydroxystearate and mixed until uniformly dispersed. One drop of clove oil (90% eugenol), or 30 μL is added to the surfactant/CLA mixture and further mixed until uniform integrated into the emulsion. This is added to 100 mL of warm water (100° F.), and mixed until clear. A water-soluble, pleasant tasting CLA liquid concentrate is produced that contains 200 mg CLA/mL. This can be added to water or a beverage to deliver 200-500 mg of CLA in an 8 oz. serving size without unpleasant taste.

What is claimed is:

1. A water-soluble formulation, comprising:
dietary fatty acid;
non-ionic surfactant; and
lipophilic taste masking agent.

2. The formulation of claim 1, wherein the formulation comprises about 40 to 80 vol % non-ionic surfactant, about 2 to 50 vol % dietary fatty acid, and about 0.001 to 1 vol % lipophilic taste masking agent, and wherein the formulation is devoid of water.

3. The formulation of claim 1, wherein the formulation consists essentially of the dietary fatty acid, the non-ionic surfactant, the lipophilic taste masking agent, and optionally, water and/or a pharmaceutically acceptable excipient.

4. The formulation of claim 1, wherein the non-ionic surfactant is admixed with the dietary fatty acid while the non-ionic surfactant is within the temperature range from 80° F. to 200° F.

5. The formulation of claim 1, wherein the dietary fatty acid is selected from one or more omega-3 fatty acids.

6. The formulation of claim 1, wherein the dietary fatty acid has a trigyceride concentration of at least 60 wt %.

7. The formulation of claim 1, solubilized in water to form a clear aqueous solution.

8. The formulation of claim 7, wherein the formulation comprises about 10 to 40 vol % non-ionic surfactant, about 0.5 to 20 vol % dietary fatty acid, about 0.0005 to 0.5 vol % lipophilic taste masking agent, and about 50 to 85 vol % water.

9. The formulation of claim 7, wherein the lipophilic taste masking agent is present in the formulation at a concentration of about 2 to 200 microliters/liter of the formulation as a whole.

10. The formulation of claim 1, wherein the non-ionic surfactant is selected from the group consisting of non-ionic water soluble mono-, di-, and tri-glycerides; non-ionic water soluble mono- and di-fatty acid esters of polyethylene glycol; non-ionic water soluble sorbitan fatty acid ester; polyglycolyzed glyceride; non-ionic water soluble triblock copolymers; glycerol-polyethylene glycol oxystearate; macrogolglycerol ricinoleate; macrogolglycerol hydroxystearate; polyethylene glycol 660 hydroxystearate; combinations thereof; and derivatives thereof.

11. The formulation of claim 1, wherein the lipophilic taste masking agent is an essential oil.

12. The formulation of claim 11, wherein the essential oil is clove oil or another essential oil that comprises eugenol.

13. The formulation of claim 11, wherein essential oil is present in the formulation at from about 5-100 ppm by volume.

14. The formulation of claim 1, in the form of a beverage, a concentrate, a spray, or topical formulation.

15. The formulation of claim 1, said formulation in a non-alcoholic form.

16. The formulation of claim 1, wherein the formulation has acceptable sensory characteristics after one week of storage at room temperature with a peroxide value of less than about 30.

17. A method of taste masking dietary fatty acid in water, said method comprising the steps of:
warming non-ionic surfactant to a temperature; and
combining dietary fatty acid with the non-ionic surfactant, lipophilic taste masking agent, and water to form stabilized, clear, water-soluble, fatty acid solution.

18. The method of claim 17, wherein the step of combining is carried out, as follows:
combining the dietary fatty acid, the non-ionic surfactant after warming, and the lipophilic taste masking agent to form a surfactant-dietary fatty acid-lipophilic taste masking agent mixture; and
combining the surfactant-dietary fatty acids-lipophilic taste masking mixture with the water.

19. The method of claim 17, wherein the formulation has acceptable sensory characteristics after one week of storage at room temperature with a peroxide value of less than about 30.

20. The method of claim 17, wherein the lipophilic taste masking agent is an essential oil.

21. The method of claim 17, wherein the non-ionic surfactant is selected from the group consisting of non-ionic water soluble mono-, di-, and tri-glycerides; non-ionic water soluble mono- and di-fatty acid esters of polyethylene glycol; non-ionic water soluble sorbitan fatty acid ester; polyglycolyzed glyceride; non-ionic water soluble triblock copolymers; glycerol-polyethylene glycol oxystearate, macrogolglycerol ricinoleate; macrogolglycerol hydroxystearate; polyethylene glycol 660 hydroxystearate; combinations thereof; and derivatives thereof.

22. A method of making a pleasant tasting and smelling, water-soluble pharmaceutical liquid composition of dietary fatty acids, comprising the steps of:
heating water-soluble non-ionic surfactant in a container to a temperature of about 90° F. to about 200° F. while mixing the surfactant until clear;
adding dietary fatty acid triglyceride to the non-ionic surfactant and stirring until thoroughly mixed so as to constitute from 0.1 wt % to 25 wt % dietary fatty acid and from 70 wt % to 99.9 wt % non-ionic surfactant, wherein the dietary fatty acid is sufficiently dispersed or dissolved in the non-ionic surfactant so that a gel composition is formed that contains no visible micelles or particles of dietary fatty acid;
dissolving lipophilic essential oil or taste masking agent in said gel composition; and
adding the gel composition containing the lipophilic essential oil or taste masking agent to warm water while continuously stirring the water until a clear solution is formed.

23. The method of claim 22, wherein the non-ionic surfactant is selected from the group consisting of non-ionic water soluble mono-, di-, and tri-glycerides; non-ionic water soluble mono- and di-fatty acid esters of polyethylene glycol; non-ionic water soluble sorbitan fatty acid ester; polyglycolyzed glyceride; non-ionic water soluble triblock copolymers; glycerol-polyethylene glycol oxystearate; macrogolglycerol ricinoleate; macrogolglycerol hydroxystearate; polyethylene glycol 660 hydroxystearate; combinations thereof; and derivatives thereof.

24. A method as in claim 22, wherein the lipophilic essential oil or taste masking agent is clove oil or another essential oil comprising eugenol.
